(19) United States
(12) Patent Application Publication
(10) Pub. No.: US 2003/0088210 A1
(43) Pub. Date: May 8, 2003

(54) Regulated gas delivery apparatus for gas-column angioscopy

(76) Inventors: Laszlo Miskolczi, Tonawanda, NY (US); James D. Flaherty, Derby, NY (US); Afshin A. Divani, Buffalo, NY (US)

Correspondence Address: David L. Principe, Hodgson, Russ, Andrews, Wood & Goodyear, LLP, One M&T Plaza, Suite 2000, Buffalo, NY, 14203-2391, US

(21) Appl. No.: 09/732,667

(22) Filed: Dec. 8, 2000

Related U.S. Application Data
(63) Continuation-in-part of application No. 09/542,193, filed on Apr. 4, 2000.
(60) Provisional application No. 60/169,893, filed on Dec. 9, 1999.

(51) Int. Cl. A61M 37/00; A61M 29/00

(52) U.S. Cl. 604/99.02; 604/26; 604/98.01

(57) ABSTRACT

An apparatus and a method for establishing a static column of gas inside a blood vessel and a system for automatically regulating the delivery and removal of the gas from the target blood vessel. The regulated gas delivery system for use with the gas-column angioscopy procedure comprises a gas reservoir, a pair of syringes operated by computer controlled electromotors, a valve system for directing the flow of gas into and out of the system, and a catheter assembly for establishing the gas-column inside the target vessel and for introducing fiber optic and microsurgical devices into the lumen of the target vessel.

### CROSS-REFERENCE TO RELATED APPLICATION

The present application is a continuation-in-part of U.S. patent application Ser. No. 09/542,193 filed Apr. 4, 2000, and entitled “Gas-Column Angioscopy”; and also claims priority to U.S. Provisional Patent Application Serial No. 60/169,893 filed on Dec. 9, 1999, and entitled “Regulated Gas Delivery Apparatus for Gas-Column Angioscopy”; both of which are incorporated herein by reference.

### FIELD OF THE INVENTION

The present invention relates generally to endoluminal angioscopic techniques and particularly to an apparatus and method for performing gas-column angioscopy and an apparatus for regulating the gas delivery for gas-column angioscopy.

### BACKGROUND OF THE INVENTION

Endovascular angioscopy has been of limited clinical utility for many reasons. Poor visual quality, brief and interrupted images, and excessive saline infusion volumes, comprise several of the problems.

Maintaining a blood-free field of observation with saline infusion typically requires large volumes of saline and sophisticated injection systems because blood quickly mixes with saline to distort the image. These saline loads can be harmful especially to patients with heart failure or renal insufficiency, and they also may cause pulmonary edema. Also, the cerebral circulation is particularly susceptible to ischemia after direct large volume saline infusion. Accordingly, there is a need for alternatives to the continuous saline infusion method.

As an alternative to saline, carbon dioxide has been used because it evacuates the blood without the mixing caused by saline. Carbon dioxide is colorless, odorless, noncombustible, and has a very low viscosity. Also, carbon dioxide can be delivered through microcatheters and angioscopic flush channels.

In the peripheral circulation, image quality comparable or superior to saline-based systems has been provided by transient carbon dioxide infusion angioscopy, e.g., renal angioscopy. Carbon dioxide has also served as a nontoxic, nonallergenic, negative contrast angiographic medium for peripheral diagnostic angiography.

In animal studies, a relatively large volume, carbon dioxide flush has been used to obtain high resolution angioscopic images. These results have proven the ability of carbon dioxide to establish a clear visual field for angioscopy. The drawback has been the volume of carbon dioxide that has to be infused, and the fact that the carbon dioxide has not been removed from the circulation after the procedure.

Animal studies in which large volumes of carbon dioxide have been injected into the cerebral circulation have disagreed as to its safety. This fear of neurotoxicity associated with carbon dioxide has prevented its use in and near the intracranial circulation.

Accordingly, carbon dioxide has been used as a clear visual medium for angioscopic imaging but has been limited by concerns related to the effects of large infusions of carbon dioxide into the blood and especially in or near the intracranial circulation. What is needed is an angioscopic technique and a regulated gas delivery system that enables prolonged angioscopic visualization without saline infusion and without the drawbacks associated with large and continuous infusions of carbon dioxide.

### SUMMARY OF THE INVENTION

The present invention meets the above described need by providing an apparatus and method for performing endovascular diagnosis and interventions with prolonged angioscopic guidance. The invention does not require continuous infusions of saline or carbon dioxide into the blood.

Generally described, the present invention provides an apparatus and a method for establishing a static column of gas inside a blood vessel. The column of gas is maintained against an occlusion balloon catheter by an anti-gravitational arterial positioning. The apparatus and method involve the infusion of a discrete amount of carbon dioxide that can be removed after the procedure. The relatively small amount of carbon dioxide required and the removal of the carbon dioxide after the procedure substantially eliminate the problems associated with the continuous infusion of carbon dioxide in large volumes. Thus, the present invention is suitable for use in both the peripheral circulatory system and in blood vessels closer to the head such as the carotid arteries.

In a preferred embodiment, a multiple lumen balloon catheter is introduced into the femoral artery percutaneously via a sheath introducer as known to those of ordinary skill in the art. Once introduced the catheter is deployed via the sheath introducer and a guide wire to a blood vessel lumen.

The position of the catheter during deployment is verified by imaging techniques such as fluoroscopy. Once the catheter reaches the lumen of the target artery, the target vessel is placed in a subhorizontal position and the balloon catheter is inflated to occlude the blood flow. Prior to inflation of the balloon, the target vessel is placed in a subhorizontal position. Next, one of the lumens of the balloon catheter is flushed with saline and then filled with carbon dioxide. The carbon dioxide is injected through the balloon lumen via a syringe until a gas column becomes visible with fluoroscopy. The volume of the carbon dioxide gas can be varied manually using a syringe or by using the regulated gas delivery apparatus of the present invention. The subhorizontal position of the artery keeps the gas buoyed against the balloon, and the pressure of the gas in the catheter stabilizes the distal gas-blood interface. In this manner, the carbon dioxide is injected through the catheter lumen into the vessel lumen under pressure control.

The carbon dioxide evacuates the blood from the targeted section of the vessel lumen and provides a region for angioscopic viewing.

A fiber optic catheter is introduced through one of the other lumens in the balloon catheter to establish angioscopic guidance in the vessel lumens, as shown in the enclosed drawing.

The carbon dioxide remains in a stable column segregated from the blood so long as the balloon is inflated. A small amount of carbon dioxide dissolves either through the endothelium or at the distal gasblood interface or both, but only balloon rupture or position change of the subject would cause the gas to escape. The preferred angle with regard to the horizontal is at least 20 degrees, as shown in the enclosed drawing. When the angle is decreased below this level, the column becomes increasingly unstable and the gas eventually escapes.

With the blood vessel occluded, the carbon dioxide column established, and the angioscope deployed; the environment is stable enough to provide for prolonged angioscopically controlled diagnostic procedures and interventions. The interventions can be performed through instruments such as scissors, forceps, and the like that are remotely controlled. The miniaturized instruments are capable of being introduced into the target area through a catheter lumen and are capable of being remotely controlled from outside of the body through mechanical or electromechanical devices as known to those of ordinary skill in the art.

The regulated gas delivery apparatus of the present invention advantageously provides precise control of gas volumes (to 0.1 ml), injection pressure and speed, gas removal, and total volumes of gas used. Also, the device adds ease of use and speed to the gas column angioscopy method. Additional benefits include a reduced risk of gas embolism and the maintenance of sterility by means of a gas filter. Also, this device may serve as an aspiration vehicle for intra-arterial debris that is created during endovascular procedures, which may not be visible by angiography.

### BRIEF DESCRIPTION OF THE DRAWINGS

The invention is illustrated in the drawings in which like reference characters designate the same or similar parts throughout the figures of which:

FIG. 1 is a diagrammatic view of the gas-column angioscopy technique of the present invention;

FIG. 2 is schematic diagram of the regulated gas delivery system of the present invention;

FIG. 3A is a plan view of a catheter assembly of the present invention;

FIG. 3B is a sectional view taken along section lines 3B-3B in FIG. 3A;

FIG. 3C is a sectional view taken along section lines 3C-3C in FIG. 3A;

FIG. 3D is a sectional view taken along section lines 3D-3D in FIG. 3A;

FIG. 4 is a schematic diagram of the stepping electromotor assembly; and, p FIG. 5 is a graph of injection speed versus time for the waveform generator assembly of the present invention.

### DESCRIPTION OF THE PREFERRED EMBODIMENTS

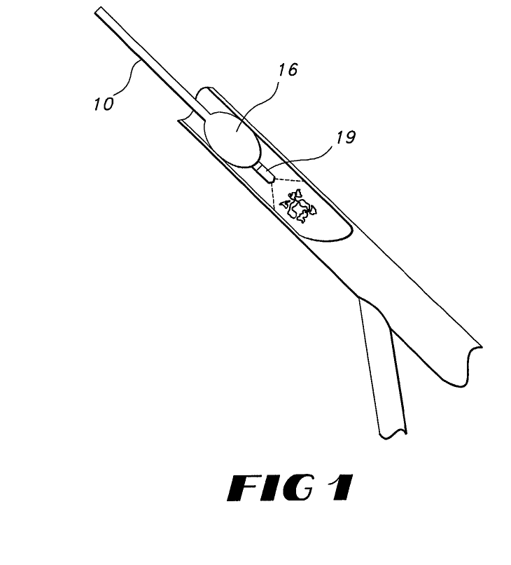

Referring initially to FIG. 1, a catheter 10 of the preferred embodiment is a multiple lumen balloon catheter that provides lumens for gas delivery, balloon inflation, introduction of fiberoptic devices, and introduction of microsurgical devices. The catheter 10 controls the placement and inflation of an occlusion balloon 16. An angioscope 19 is preferably of the fiber optic type, but also can be a single CCD device mounted on the tip of a flexible wire. In either case the angioscope 19 can also be mounted onto the balloon catheter 10 itself, eliminating the need for an extra lumen.

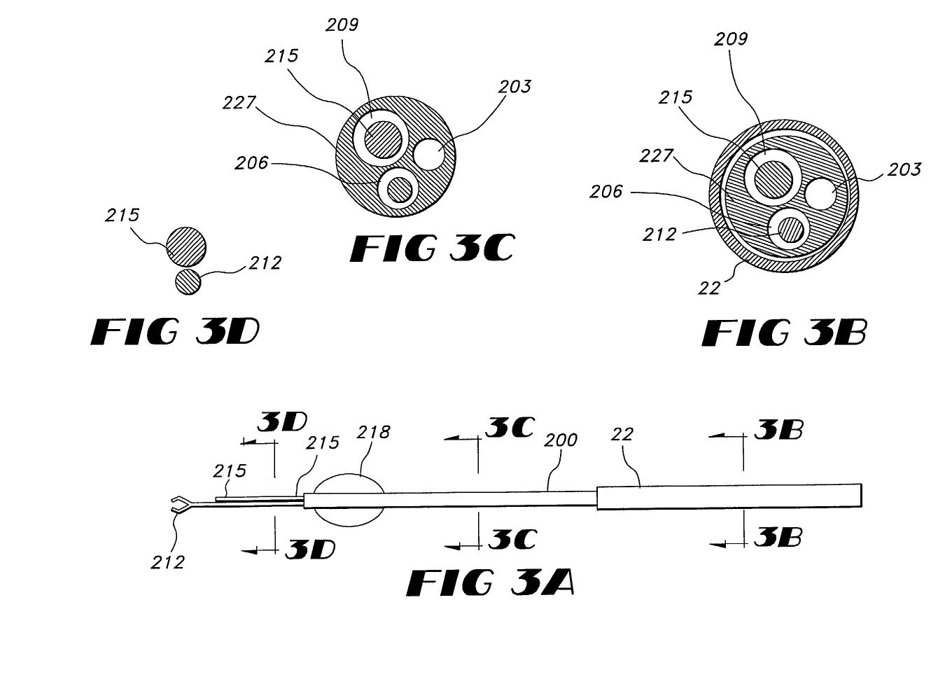

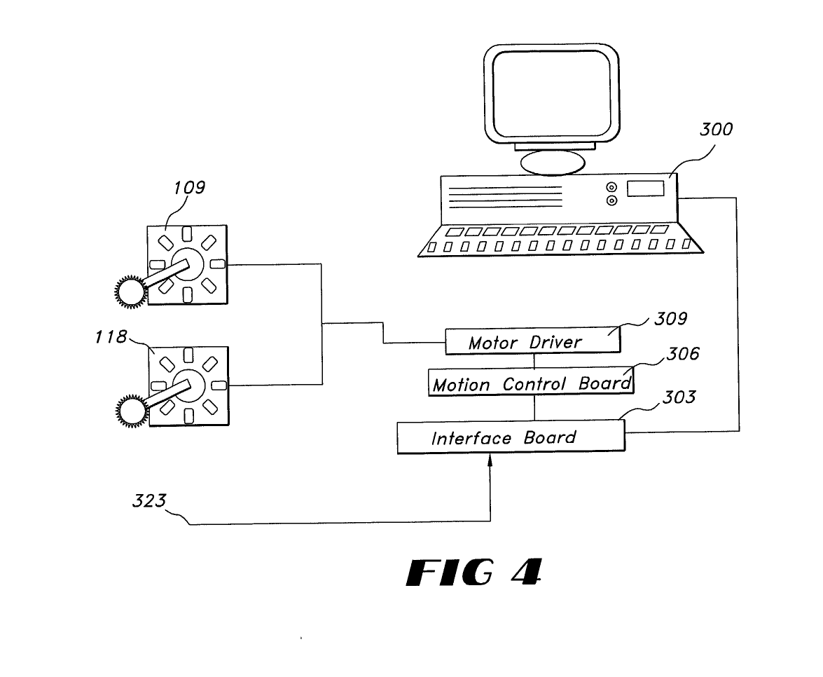

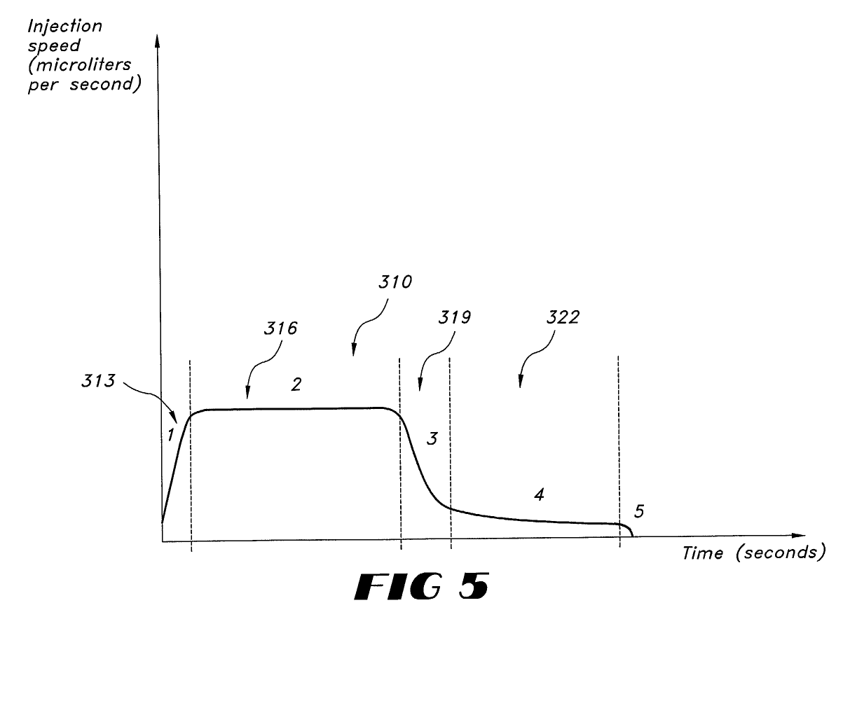

With the aid of a guide wire and an introducer sheath 22 (shown in FIG. 3a), the balloon catheter 10 is deployed to the target blood vessel. Once the target portion of the blood vessel is reached by the balloon catheter 10 as confirmed by fluoroscopic imaging, the blood vessel is placed in a subhorizontal position and the balloon 16 is inflated to occlude the blood flow. Depending on the anatomic location of the balloon 16 and/or the characteristics of the patient, the blood flow may be occluded for a prolonged period of time. In areas where natural bypass occurs, such as the Circle of Willis, prolonged occlusion may be feasible in most patients.

Next, the tip of the angioscope 19 is extended beyond the balloon 16 for approximately 0.5-1 mm. One of the lumens of the balloon catheter 10 is then flushed with saline and filled with approximately 0.5-2 cc's of carbon dioxide. The 0.5-2 cc of CO₂ is injected into the vessel lumen under pressure control by a syringe. The CO₂ evacuates the blood and establishes a column inside the vessel lumen where blood and gas are segregated. The CO₂ in the syringe provides support to the distal gas-blood interface and allows modification of the CO₂ column. In this manner, the carbon dioxide provides a light conducting media for prolonged visualization within a blood vessel.

As discussed above, the balloon 16 is positioned higher than the column of CO₂ such that the minimum angle with respect to the horizontal is approximately 20 degrees. The column of carbon dioxide is trapped by the blockage of proximal blood flow with the balloon 16 and by positioning the target artery subhorizontally. At some point below 20 degrees, the column destabilizes and the CO₂ will escape distally.

Once the angioscope is in position and the CO₂ column is stable, interventions may be performed within the CO₂ column. Devices such as scissors, forceps, stents, various blades for cutting, needles, drills, laser devices, ultrasonic devices, infrared or ultraviolet light conducting or emitting probes, and the like, mounted on flexible wires for introduction through a catheter can be introduced into the vessel lumen through catheter. Because of the high degree of stability and visibility created in the vessel lumen; endovascular interventions can be performed with a degree of angioscopic guidance that has not been possible prior to applicant's invention.

After the procedure has been completed, the remaining carbon dioxide can be removed from the blood vessel lumen with the syringe rather than having it released into the blood.

The syringe may provide for manual control of the pressure of the carbon dioxide inside the blood vessel. As an alternative, the regulated gas delivery system described below provides for automated control of the gas delivery. The syringe also provides for extracting the carbon dioxide from the blood vessel after the procedure is completed.

The second lumen provides access for the instruments and for other catheters. The instruments are mounted to the tip of a flexible wire. At the opposite end of the wire, a pistol grip actuator may provide for control of the instrument during the intervention. Other mechanical and electromechanical control devices and the like would also be suitable.

Additional ports having valves are also provided for irrigating the introducer sheath (which is normally deployed to the abdominal aorta or iliac arteries) and for introducing a mixture of radiopaque contrast material and saline for better X-ray visualization of the balloon 28 during initial deployment.

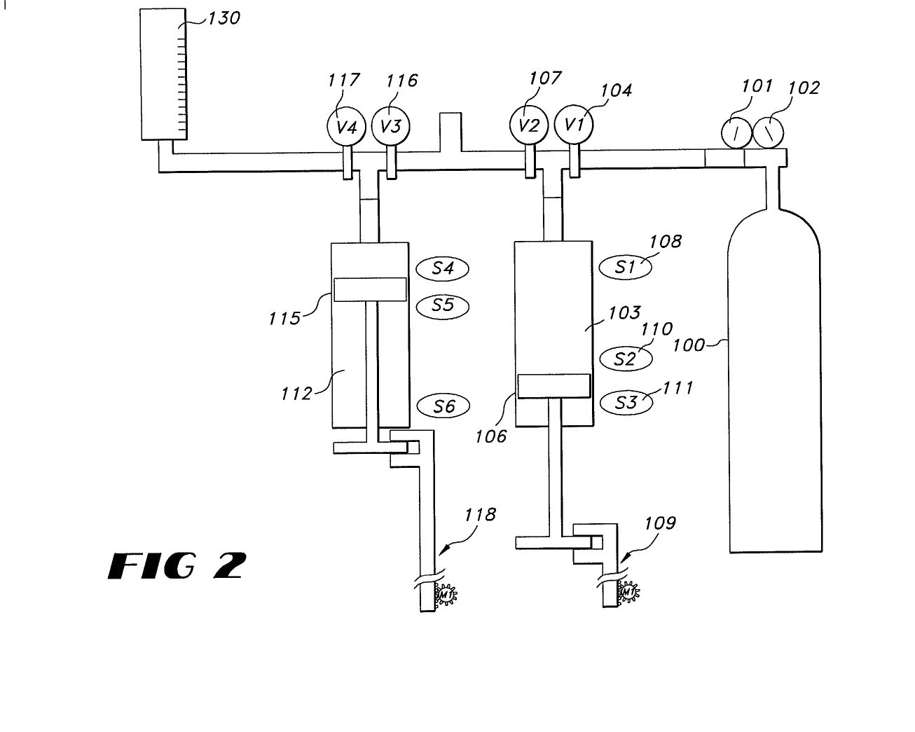

Turning to FIG. 2, the regulated gas delivery system of the present invention is shown. A canister 100 containing a supply of gas is equipped with a sterility filter 101 and a pressure gauge 102. The canister 100 is connected to an injection syringe 103. An electronic valve 104 is disposed between the canister 100 and the injection syringe 103. A plunger/piston assembly 106 of the injection syringe 103 is controlled by a stepping electromotor assembly 109 for intake from the canister 100 and discharge through the catheter assembly (shown in FIG. 3a) into the target blood vessel. A second valve 107 is disposed between the injection syringe 103 and the catheter assembly. The injection syringe 103 has a plurality of electro-optical position sensors 108, 110, and 111 for determining the position of the plunger/piston 106. In operation, valve 104 opens to allow a charge of gas to enter the injection syringe 103. The plunger/piston 106 is retracted by the stepping electromotor 109 until it reaches a certain sensor position (111) and sufficient time has passed for the gas to flow into the injection syringe 103 from the canister 100. Next, valve 104 is closed and valve 107 is opened. With valve 107 open, gas from the injection syringe 103 can be delivered in a regulated fashion to the target blood vessel. The electro-optical sensors 108, 110, and 111 determine when the supply of gas is depleted in the injection syringe 103 and needs to be recharged. Syringe 103 is recharged if: a) Valve 107 is closed and the piston has passed the sensor 110 (optional refill); or, if b) Valve 107 is open (syringe is in use and inflating) and the piston reaches the sensor 108 (forced refill). The stepping electromotor assembly 109 provides for precise control of the syringe 103 and the resulting injection speeds and volumes according to the waveform generator assembly (shown in FIG. 5). The waveform generator assembly provides several advantages over the manual techniques. To manually create a gas column in a target arterial segment, a small amount of gas must be introduced through the balloon catheter lumen via a handheld syringe followed by saline until the gas is visible beyond the balloon tip on fluoroscopy. This method, although effective is burdensome for the operator and does not permit precise control of the gas column length, both at the initiation point of the column and during an imaging session. The waveform generator of the present invention facilitates the automatic establishment of the gas column at a desired length. The wavelength operates using precalculated volumes specific for the balloon catheter and introducer sheath assembly chosen, which are entered into the computer. The desired gas column length selected by the operator is visualized on fluoroscopy and the clear imaging medium is seen on a video screen connected to the angioscopic catheter. Once established, the operator can adjust the column length using a manual mode on the electromotor, which controls supplemental gas injection and gas removal.

A second syringe 112 for suction is connected in parallel to the first syringe 109 and also has a pair of electronically controlled valves 116 and 117. The plunger/piston 115 of the second syringe is also controlled by a stepping electromotor assembly 118. In order to remove the carbon dioxide after the procedure, valves 116 and 117 are operated in connection with syringe 112. A volume gauge 130 may also be used to determine the volume of fluid that is removed.

Turning to FIG. 3A, the catheter shaft assembly 200 of the present invention includes lumens 203, 206, and 209 for the balloon 218, the gas, the microsurgical instruments 212 and for the fiberoptic devices. As shown the microsurgical instruments 212 and the fiberoptic catheter 215 extend beyond the balloon 218 into the target vessel.

In FIG. 3B, the introducer sheath 22 and balloon catheter 224 are shown in cross-section. The balloon catheter 224 includes lumens 203, 206, and 209 for the fiberoptic catheter 215, for balloon 218 inflation, and for the microsurgical devices 212. A lumen is provided between the balloon catheter wall 227 and the introducer sheath 22 for irrigation of the introducer sheath.

In FIG. 3C, the balloon catheter 224 extends beyond the introducer sheath 22 into the target vessel. In operation the blood vessel is occluded by inflation of the balloon 218 and the gas column is established by injecting gas through one of the lumens in the balloon catheter 224.

In FIG. 3D, the microsurgical instruments 212 and the fiberoptic catheter 215 extend beyond the balloon catheter 224 and into the gas column such that visualization inside the target vessel as well as microsurgical procedures can occur.

Turning to FIG. 4, the stepping electromotor controls are shown schematically. The stepper motors 109 and 118 are controlled by motor drives which are controlled by a microprocessor 300 that provides for precise controls of the motors 109, 118 such that precise amounts of gas can be delivered through the injection syringe 103. The central processor 300 also receives input signals 323 from the electro-optical sensors and makes adjustments accordingly.

The microprocessor 300 controlled electromotors 109 and 118 are controlled through an interface board 303, a motion control board 306 and motor drives 309. The motors are controlled based on a waveform for injection speed versus time that is generated based on precalculated volumes for the amount of gas for the gascolumn and the amount of gas that can be held in the balloon catheter 224.

As shown in FIG. 5, the electromotors are controlled according to a waveform 310 that provides for an initial rapid acceleration phase 313 and then a sustained high speed injection phase 316 where the catheter assembly is being filled with gas. The initial phases are followed by a rapid deceleration phase 319 which occurs once the catheter 224 is filled with gas and the gas column is beginning to be established inside the target blood vessel. During the next phase 322 for the establishment of the gas column a sustained low speed injection rate is maintained. Finally, once the gas column is fully established the gas inflow stops, and the control is switched to a manual or standby mode where the system remains unless additional gas is needed to compensate for losses of gas through the endothelium or at the distal gas-blood interface or both.

Accordingly, the present invention offers several advantages. Direct visualization of the endoluminal surface by angioscopy is an established tool in vascular procedures. In the coronary arteries, angioscopy has been used in all phases of lesion stenting. It has also been useful to distinguish thrombotic from nonthrombotic occlusions. Angioscopy may be useful in determining the need for additional stents or thrombolytic therapy and in predicting restenosis. It has been said to be superior to angiography and IVUS for the depiction of thrombi, dissection and friable plaques in venous grafts. The present invention facilitates the visualization of endoluminal surfaces by providing a stable visual field for prolonged viewing.

Carotid revascularization with angioplasty and stent procedures is emerging as a safe and effective, but much less invasive, alternative to endarterectomy for select patients. The present invention provides a powerful tool for carotid and peripheral revascularization by showing diseased segments and by providing angioscopic guidance for wires, stents, and other endovascular devices. The present invention provides for much longer periods of viewing than the saline method. The prolonged occlusion is feasible where blood supply to the occluded territory of the brain is maintained via collateral flow from the Circle of Willis.

Also, carbon dioxide is a safer and more effective medium than saline for the laser ablation of atherosclerotic plaques. Gas column angioscopy could guide laser angioplasty, which has failed due largely to the inability to direct the beam.

Also, the present invention is useful for the accurate identification of carotid plaque ulceration which may be an important step in stroke prevention.

The present invention has wide application to a large array of endovascular devices, and the present invention could be used globally in the vascular system.

The regulated gas delivery system of the present invention also provides several advantages. The ability to regulate the amount of gas introduced in the system provides for lower volumes of gas used per imaging session and also over the course of an entire procedure (if multiple imaging sessions are desired), and therefore the risk of gas embolism is reduced. Also, the apparatus both injects and removes gas from the target artery. The initial gas injection is governed by the specific waveform pattern. And the synchronization of injection and removal of gas provided by this automated system permits quick and easy repeat imaging sessions without occluding the target vessel for an extended period of time.

While the invention has been described in connection with certain preferred embodiments, it is not intended to limit the scope of the invention to the particular forms set forth, but, on the contrary, it is intended to cover such alternatives, modifications, and equivalents as may be included within the spirit and scope of the invention.

What is claimed is

1. A fluid delivery apparatus for gas-column angioscopy, comprising:
a fluid reservoir;
a first syringe in fluid communication with the gas reservoir and having a first plunger;
a balloon catheter having multiple lumens and disposed in fluid communication with the first syringe;
a second syringe in fluid communication with the balloon catheter and having a second plunger;
a drive system adapted to drive the first and second plunger in a first direction and a second direction opposite the first direction;
at least one first valve disposed between the gas reservoir and the first syringe;
at least one second valve disposed between the first syringe and the balloon catheter; and,
at least one third valve disposed between the balloon catheter and the second syringe.

2. The gas delivery apparatus of claim 1, wherein the drive system comprises at least one motor and drive attached to the first plunger and second plunger.

3. The gas delivery apparatus of claim 1, wherein a gas comprising carbon dioxide is contained in the fluid reservoir.

4. The gas delivery apparatus of claim 1, wherein the drive system comprises at least one stepping electromotor.

5. The gas delivery apparatus of claim 1, further comprising at least one position sensor disposed on the first syringe.

6. The gas delivery apparatus of claim 1, wherein the apparatus further comprises a volume gauge.

7. A regulated gas delivery apparatus for gas-column angioscopy, comprising:
at least one catheter having an inflatable occluding balloon carried thereby;
an angioscopic medium adapted to be deployed through the at least one catheter into a target area of a blood vessel to establish a column of angioscopic medium for angioscopic viewing;
an angioscope adapted to be deployed through the at least one catheter into a target area of a blood vessel to establish a field of view inside the column of the angioscopic medium, the angioscope adapted to transmit images from the inside of the target area of the blood vessel;
a fluid reservoir;
a first syringe in fluid communication with the fluid reservoir and having a first plunger;
a second syringe in fluid communication with the catheter and having a second plunger;
a drive system adapted to drive the first and second plunger in a first direction and a second direction opposite the first direction;
at least one first valve disposed between the fluid reservoir and the first syringe;
at least one second valve disposed between the second syringe and the balloon catheter; and,
at least one third valve between the balloon catheter and the second syringe.

8. The gas delivery apparatus of claim 7, wherein the drive system comprises at least one motor and drive attached to the first plunger and second plunger.

9. The gas delivery apparatus of claim 7, wherein a gas comprising carbon dioxide is contained in the fluid reservoir.

10. The gas delivery apparatus of claim 7, wherein the drive system comprises at least one stepping electromotor.

11. The gas delivery apparatus of claim 7, further comprising at least one positon sensor disposed on the first syringe.

12. The gas delivery apparatus of claim 7, wherein the apparatus further comprises a volume gauge.

13. A gas delivery apparatus for gas-column angioscopy, comprising:
means for storing a fluid;
means for injecting a fluid through a catheter into a target area of a blood vessel;
means for suctioning fluid through the catheter from the target area of the blood vessel;
means for driving a plunger in the injecting means;
means for driving a plunger in the suctioning means;
first valve means for controlling flow of the fluid between the storing means and the injecting means;
second valve means for controlling the flow of the fluid between the catheter and the suctioning means; and,
third valve means for controlling the flow between the injection means and the catheter.

14. A method of regulating the delivery of a fluid, comprising:
opening a first valve between a fluid reservoir and a first syringe;
retracting a plunger in the first syringe to fill the syringe;
closing the first valve and opening a second valve disposed between the first syringe and a balloon catheter;
injecting a fluid through the balloon catheter into a target area of a vessel at a rate predetermined by a waveform for gas column angioscopy;
closing the second valve;
opening a third valve disposed between the balloon catheter and a second syringe; and,
suctioning the balloon catheter with the second syringe such that the fluid is removed from the target area.
